(19) United States
(12) Patent Application Publication
(10) Pub. No.: US 2024/0269458 A1
(43) Pub. Date: Aug. 15, 2024

(54) SYSTEM AND METHOD FOR UTILIZING ANTEGRADE SHEATHS

(71) Applicant: ABIOMED, Inc., Danvers, MA (US)

(72) Inventors: Mithun Rajaram, Danvers, MA (US); Glen Fantuzzi, Danvers, MA (US); Christopher Korkuch, Danvers, MA (US); Ying Xu, Danvers, MA (US); Matthew D'Agostino, Danvers, MA (US); Robert Fishman, Danvers, MA (US)

(73) Assignee: ABIOMED, Inc., Danvers, MA (US)

(21) Appl. No.: 18/438,331

(22) Filed: Feb. 9, 2024

Related U.S. Application Data
(60) Provisional application No. 63/444,536, filed on Feb. 9, 2023.

(51) Int. Cl. A61M 60/865; A61M 25/00; A61M 25/01; A61M 25/06; A61M 39/06

(52) U.S. Cl. CPC A61M 60/865; A61M 25/0023; A61M 25/0068; A61M 25/0097; A61M 25/0606; A61M 39/06; A61M 2025/0037; A61M 2025/018; A61M 2209/00; A61M 2210/12

(57) ABSTRACT

Various systems and devices, such as sheaths configured for antegrade and retrograde insertion into a patient's vasculature, are provided, to aid in preventing limb ischemia resulting from use of mechanical circulatory support. Such systems and devices may include various combinations of hubs, sheaths, and needle designs to allow, e.g., multiple medical devices to be inserted into a patient's vasculature, while minimizing blockage of the blood vessels.

### CROSS-REFERENCE TO RELATED APPLICATIONS

The present application claims priority to U.S. 63/444,536, filed Feb. 9, 2023, the contents of which are incorporated by reference herein in its entirety.

### TECHNICAL FIELD

The present disclosure relates to methods, devices, and systems used in the prevention of, e.g., limb ischemia resulting from use of mechanical circulatory support.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Intravascular medical devices such as percutaneous pumps (e.g., IMPELLA® heart pumps by Abiomed, Inc., Danvers, Massachusetts), catheters, guidewires, balloon angioplasty catheters, delivery sheaths, and implant delivery systems may be used during minimally invasive procedures in the cardiovascular, cerebrovascular and peripheral vascular systems. Such medical devices can be introduced into a patient in various ways. In one common approach, pump assemblies are inserted by a catheterization procedure through the femoral artery using a sheath system which may remain in place through the duration of use. In other scenarios, the sheath system used to facilitate placement of the pump may be peeled away off of the pump catheter, with a secondary sheath assembled on the catheter advanced into the vasculature system for long term access site management (e.g. hemostasis, pump stability).

### BRIEF SUMMARY

In various aspects, a retrograde sheath may be provided. The sheath may include a hub. The hub may include a first opening extending from a proximal end to a distal end of the hub. The hub may include a second opening extending from the proximal end to the distal end of the hub. The sheath may include a first cannula. The first cannula may be coupled to a distal end of a hub. The first opening may be operably coupled to the cannula. The second opening may include a membrane or hemostasis valve. The first opening may have a larger diameter at the proximal end, such that the second opening connects to the first opening within the proximal hub at a point between the distal end and the proximal end. The first opening may be configured to be directed towards a blood vessel, and the second opening may be configured to be directed towards the same blood vessel. The first cannula may be configured to be directed into the blood vessel in a first direction, and the second cannula may be configured to be directed into the blood vessel in a second direction different from the first direction. The first cannula may have a larger diameter at a proximal end than a distal end. The proximal end may include a first lumen that extends a first length towards the distal end (such as the full length of the cannula) and a second lumen parallel to the first lumen that extends towards the distal end a distance less than the first length.

In various aspects, a retrograde sheath may be provided. The sheath may include a hub. The hub may include a first opening extending from a proximal end to a distal end of the hub. The sheath may include a distal cannula having a distal cannula body coupled to a distal end of a hub. The first opening may be operably coupled to the distal cannula. The sheath may include an accessory coupled to a proximal end of the distal cannula body or to the hub, the accessory including a separate cannula body. The distal cannula may be configured to be directed towards a blood vessel, and the separate cannula body may be configured to point towards the same blood vessel. The accessory may be removably coupled to the distal cannula body or the hub. The accessory may be permanently coupled to the distal cannula body or the hub.

In various aspects, a retrograde sheath may be provided. The sheath may include a proximal hub coupled to a cannula body. The retrograde sheath may include an adjustable portion. The adjustable portion may include a distal end of the proximal hub and/or a proximal end of the cannula body. The adjustable portion may be located within the distal end of the proximal hub. The adjustable portion may be coupled between the distal end of the proximal hub and the proximal end of the cannula body. The adjustable portion may include an articulating joint. The adjustable portion may be configured to allow only 1-D rotation. The adjustable portion may be configured to allow only 1-D or 2-D rotation. A distal end of the cannula body may be oriented to face a proximal direction. An outer surface of a distal end of the cannula body, after a 180-degree bend, may be separated from an outer surface of a middle portion of the cannula body by a distance of 0.25-1 mm.

In various aspects, an introducer sheath may be provided. The sheath may include a tubular body. The tubular body may include a first lumen providing a first path extending from a proximal end of the tubular body to a distal end of the tubular body. The distal end of the tubular body may be configured to produce a micropuncture through a blood vessel. The distal end of the tubular body may be configured have an outer diameter of no more than 21 Fr. The tubular body may include a proximal portion defining a second lumen providing a second path extending from the proximal end of the tubular body and extending distally. The second lumen may have an opening at a distal end of the proximal portion configured to be positioned outside a blood vessel into which the first lumen is inserted. The second lumen may be configured to slidably receive a curved needle or wire such that the blood vessel may be entered at a different location from where the first lumen enters the blood vessel. The sheath may include a hub operably coupled to the tubular body. The hub may include a luer connection. The hub may include one luer connection operably coupled to each lumen in the tubular body. The hub may include a first opening configured to be coupled to a proximal end of the first lumen. The hub may include a second opening configured to be coupled to a proximal end of a second lumen. The first opening may be configured to slidably receive one or more medical devices configured to be inserted into a blood vessel in a first direction. The second opening may be configured to slidably receive one or more medical devices configured to be inserted into the blood vessel in a second direction different from the first direction.

In various aspects, a system may be provided. The system may include a needle operably coupled to a hub or handle. The system may include a device including a proximal hub coupled to a proximal end of a distal tubular member. The distal tubular member may include a distal tip that has curved to face a different direction from a middle portion of the distal tubular member. The system may include a catheter having a hub coupled to a distal tubular member, the catheter being configured to slidably receive the needle, and, after the needle has been removed, slidably receive the device. The needle may be configured to puncturing a blood vessel at multiple locations. The distal tip may have a fixed separation from the middle portion. The distal tip may have a variable separation from the middle portion. The needle may be a twin needle. A first needle of the twin needle may be configured to provide an access point for an antegrade sheath. A second needle of the twin needle may be configured to provide an access point for a retrograde sheath.

In various aspects, a device may be provided. The device may include a first needle, a second needle, and a proximal hub. The first needle may define a first lumen extending from a distal end to a proximal end of the first needle. The first needle may have a first length from the distal end to the proximal end. The second needle may define a second lumen extending from a distal end to a proximal end of the second needle. The second needle may have a second length from the distal end to the proximal end. The first needle and the second needle may be connected to the proximal hub. The proximal hub may have a first opening and a second opening. Each opening may extend from a proximal end to the distal end of the proximal hub. The first opening may be operably coupled to the first lumen. The second opening may be operably coupled to the second lumen. The first length may be longer than the second length. The first length may be equal to the second length. Each opening and lumen may be configured to slidably receive one or more medical devices.

In various aspects, a needle may be provided. The needle may include a sidewall that defines a first lumen extending from a proximal end to a distal end of the needle and a second lumen extending from a proximal end to an exit at an intermediate portion of the sidewall. The exit may be configured to be a distance from the distal end of the needle such that both the distal end and the exit would be positioned within a blood vessel in use.

In various aspects, a device may be provided. The device may include a hub coupled to at least one tubular member defining a first lumen and a second lumen. The at least one tubular member may extend distally away from the hub. The first lumen may be configured to slidably receive a medical device. The device may include one or more valves disposed within the hub. The hub may define a volume of space coupled to the first lumen and the second lumen, such that a path from a distal opening for the first lumen to a distal opening for the second lumen may be formed by passing through the volume of space. The first lumen may be configured such that there is excess space within the first lumen after a medical device has been inserted.

In various aspects, a reverse needle may be provided. The reverse needle may include one or more sidewalls that define a lumen extending from a proximal end towards a distal end of the reverse needle. The lumen may be shaped so as to exit via an opening on a side of the reverse needle proximal to the distal end.

In various aspects, a device may be provided. The device may include a tubular member having a proximal end coupled to a distal end of a proximal hub. The tubular member may have one or more areas, less than an entire area of the tubular member, that are puncturable by a distal end of a medical device. The one or more areas may be free of a reinforcing coil. The one or more areas may have a sidewall thickness that is thinner than a sidewall thickness at a different location. The one or more areas may be laser cut and then filled with a filler material. The filler material may be a polymer. The filler material may be different from a polymer forming a sidewall in a different location. The one or more areas may be in a portion of the tubular member, such that in use, when the tubular member is positioned correctly, the one or more areas are within a blood vessel. Each of the one or more areas may be located between a distal and proximal end of the tubular member. The one or more areas may be configured such that a medical device pass through the one or more areas and extend in an opposite direction in a blood vessel relative to a direction the tubular member extends.

In various aspects, a device may be provided. The device may include a tubular member coupled to a proximal hub. The tubular member may include a first lumen extending from the proximal hub to a distal end, and a second lumen extending from the proximal hub and exiting a side opening proximal to the distal end. A first opening in the proximal hub may be configured to slidably receive a first medical device, the first opening being operably coupled to the first lumen. A second opening in the proximal hub may be configured to slidably receive a second medical device, the second opening being operably coupled to the second lumen. At least a portion of the second medical device may have a memory shape, such that when it exits out the side opening, it returns to its normal shape. In use, when the tubular member is correctly placed, the side opening may be configured to be positioned within a blood vessel. The tubular member may include an expandable sleeve through which a second medical device is inserted. The tubular member may be a catheter, cannula, or needle. A sheath and/or sleeve may be disposed over or around any needle.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 illustrates antegrade and retrograde sheaths inserted into a patient's vasculature.

FIGS. 2-12 are illustrations of various embodiments of systems.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

For purposes of the present disclosure, a “distal” element is the part of the element located farthest away from the clinician and “proximal” is the part of the element that is closest to the clinician. In the case of a sheath deployed inside a patient, the sheath end that is deployed inside the body of the patient is the “distal” end, whereas the handle or end held by the clinician located outside the patient is the “proximal” end.

As will be appreciated, a sheath may be inserted into a patient's vasculature, such as the femoral artery, through an arteriotomy (i.e., an access site in the artery) to create an insertion path for a medical device such as a pump assembly. A portion of the pump assembly may then be advanced through an inner lumen of an initial access sheath (also referred to as an introducer sheath) and into the artery. As will be appreciated, the pump may be placed through the introducer sheath, which remains in place through the full duration of support (e.g. both in the catheterization lab and in the ICU). In some embodiments, a repositioning sheath may be advanced over the pump assembly and into the arteriotomy and remain with the patient for long term access site management (e.g. hemostasis, pump stability).

As will be appreciated, the introducer sheath must be large enough in diameter to accommodate the intravascular device, e.g., the blood pump. In some embodiments, the motor diameters of such pumps may be between 12 FR and 21 Fr or larger, , depending on the type of device. In such embodiments, the sheaths must be 13-22 Fr or larger, depending upon the pump sizes.

During the use of extracorporeal membrane oxygenation (ECMO), cannula devices are used to gain access into the patient vasculature which are the conduits for the blood being pumped by the ECMO circuit to either exit or enter the body. These cannulas may be similar to introducer sheaths and can have sizes that range from 13 F to 30 F depending on the clinical application. These diameters may also be restrictive to blood flow in a similar fashion to introducer sheaths.

While existing introducer sheaths are generally functional for device insertion, they have drawbacks. For example, due to its size, the introducer sheath can block the cross-sectional area of the artery, and thereby occlude the artery, which can drastically restrict down-stream blood flow. Restricted blood flow can be problematic for a number of reasons, including causing intermittent claudication, leg numbness/weakness and limb ischemia which may even result in loss of the limb.

Limb ischemia is a rapid and sudden decrease in limb perfusion often threatening limb viability. It may occur as a result of blockage of blood due to an indwelling sheath and/or catheter, local occlusion (e.g., atherosclerotic narrowing), and/or continuous occlusion resulting from small vessels and/or large sheaths. It may also occur as a result of a closure issue. Conventionally, limb ischemia is known as a “distal perfusion” issue in the sense that it often occurs downstream from the insertion site of the device.

For relatively large medical devices (e.g., with a maximum outer diameter equal to or greater than 12 Fr), the introducer sheath (or repositioning sheath) used in combination with the medical device may have an outer diameter which is substantially similar in size to an inner diameter of the artery or vessel in which the sheath is positioned. This match between the outer diameter of the sheath and inner diameter of the artery or vessel prevents blood from flowing from a location upstream of the arteriotomy to a location downstream of the arteriotomy. Moreover, for these relatively large medical devices, the introducer sheath (or repositioning sheath) used in combination with the medical device also requires a large access site, which may be difficult to close, or to control bleeding at when inserting or withdrawing the sheath. The size of a medical device (e.g., relatively large, or relatively small), and accordingly whether an introducer sheath or repositioning sheath is considered relatively large, can depend on a particular patient's anatomy.

Other drawbacks may include excessive bleeding at the arteriotomy during insertion or removal of the sheath, which can result in blood loss for the patient, and weakening of the patient. This is normally treated by using closure devices such as collagen injections, sutures or staples. But these types of closure devices can be challenging to use when blood flow has not yet been adequately stopped, and floods the access site being closed. Moreover, these types of closure devices generally require additional tools or steps and therefore require time to effectively close.

The size of an introducer sheath and its impact on the patient may be complicated by patient anatomy (e.g., heavy stenosis and small vessels) and condition (e.g., shock, vasospasm, patient on vasopressors with constricted vessels) which can limit the size of introducer that can be used to gain access. In the case of vasospasm, even an appropriately sized sheath to the original vessel diameter will fill the entire contracted vessel. Accordingly, for some patients a very small sheath, e.g., 6 Fr, can be too large and lead to the same occlusion and access bleed concerns as a 12 Fr sheath in another patient.

In general, the drawbacks noted above may be pronounced for long-term vascular procedures. Certain procedures in the catheterization lab are short term and therefore present less risk, for example, typically only require at most 4-5 hours, with physicians periodically checking for distal (limb) perfusion. However, even in short-term situations, physicians may forget to confirm the patient has adequate distal perfusion from the device, or miss warning signs of inadequate limb perfusion. Additionally, certain patients may be transferred to the ICU with introducer sheaths or repositioning sheaths staying in the patient for longer periods of times ranging from 1-14 days in such instances, the problems of vessel occlusion and associated limb ischemia, and access site bleeding can be exacerbated.

To overcome the problems associated with conventional techniques, retrograde and antegrade cannulation is a standard practice. However, placement of the antegrade sheath can be challenging. While placement of antegrade sheaths is challenging in general and only select physicians have the skillset to do so; placement of an antegrade sheath after the insertion of a large bore sheath or cannula (e.g., for Impella or ECMO) is even more challenging. In these clinical scenarios the blood flow is blocked by the sheath or cannula and device hubs remain outside the skin. These issues make identifying the correct anatomic location of artery challenging by pulse (no blood flow means no pulse) or by ultrasound (devices are in the way and prevent easy use of the ultrasound probe).

It may be desired to place the antegrade sheath even after you insert the intravascular device (e.g., an IMPELLA® heart pump) via a retrograde sheath. One challenge is because the retrograde sheath is typically in the way, but other difficulties include a need to use ultrasound to place it (as various medical devices are already inserted), a physician will need to switch hands to perform the insertion, and even when done well, a “dead space” may be formed between the entry site in the vessel for the retrograde sheath and the entry in the vessel for the antegrade sheath.

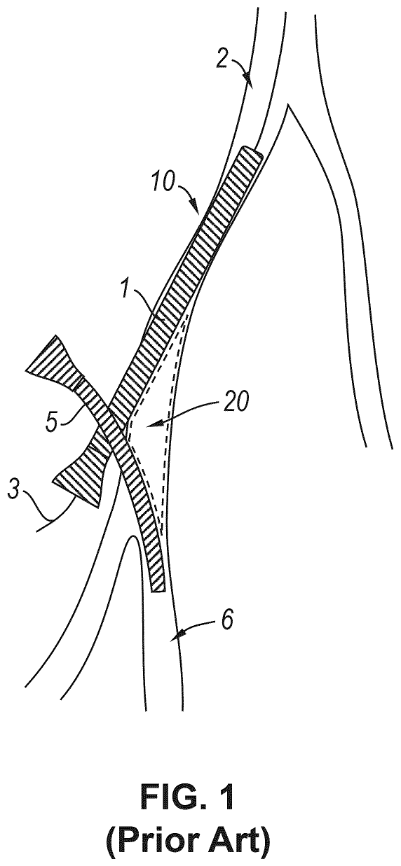

This is illustrated in FIG. 1. There, a retrograde sheath 1 can be seen, inserted into a first part 2 of a patient's vasculature, with a guidewire 3 passing through the sheath. An antegrade sheath 5 is shown, inserted into a second part 6 of the patient's vasculature. As can be seen, there is a blockage 10 in the first part of the patient's vasculature, preventing blood from flowing freely past the retrograde sheath. This, in turn, leads to a dead zone 20 in the vasculature that may be formed in some or all the volume of space within the vasculature between the antegrade and retrograde sheaths.

To improve upon these conventional techniques, various options have been be considered by the inventors and disclosed herein

In some embodiments, a feature may be added to a hub to provide an insertion angle and/or needle angle that may allow for easy access to a blood vessel, such as a femoral artery. In some embodiments, a sheath may be provided that may include a distal cannula body and a proximal hub. The proximal hub may include a first opening at a distal end, extending to the proximal end, that is operably connected to the distal cannula body.

The cannulas described herein typically include a lumen extending from a distal end to a proximal end. In some embodiments, the cannula may consist of a coil of shape memory material, with the coil being disposed over the outer diameter of the inner layer. The cannula may further have an inner layer and a thermoformed outer layer disposed over the coil and extending along the length of the cannula. In certain implementations, the coil is embedded in the outer layer. In a further embodiment, the shape memory material comprising the coil is at least one of nitinol or a copper-aluminum alloy. The outer layer of the cannula may include an outer material. In certain embodiments, the outer material may include polyurethane. In some implementations, the outer material may include Texin®. Texin is a thermoplastic polyurethane (TPU). In some implementations, the inner material may include polyurethane. In another implementation, the inner material may include Texin®. When polyurethane is used for the inner and/or outer layers, it may include polyethers or polyesters. In certain implementations, the inner and outer materials may be the same.

The hubs described herein may include ethylene vinyl acetate (EVA); styrene butadiene copolymer (SBC); styrene ethylene butylene styrene (SEBS); a high-density polyethylene (HDPE) material; a medium-density polyethylene (MDPE) material; a low-density polyethylene (LDPE) material; polyether ether ketone (PEEK); a polyether block amide; an elastomer; synthetic rubber; a polyethylene, polyurethane, or polycarbonate material with an elastic modulus of about 40 ksi, or a combination thereof. The hubs described herein also may include polyamide (Nylon), acrylonitrile butadiene styrence (ABS), and/or polyether block amide (PEBA).

The proximal hub may also include a second opening at the distal end, offset from the first opening and extending at least partially towards the proximal end, the second opening forming a hole, channel, or guide in the hub that points, along a path, towards a blood vessel the cannula will be positioned within, the path being offset and parallel to the distal cannula body, so as to provide a second entry point, different from the entry point of the cannula, into the blood vessel.

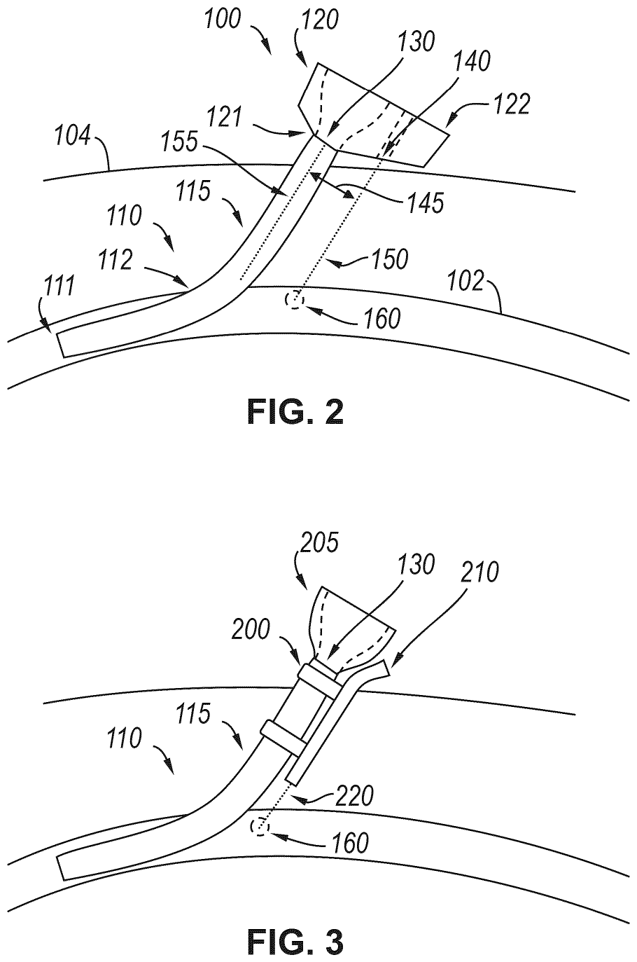

An example of this may be seen in FIG. 2. There, a sheath 100 is shown with a distal cannula body 110 coupled to a distal end 121 of a proximal hub 120. The distal end 111 of the cannula body is positioned within a blood vessel 102 of a subject 104. Proximal hub may have a first opening 130 that extends from the distal end to the proximal end, the first opening being operably coupled to the cannula body. In this way, a medical device, such as an intravascular pump, may be extended through the first opening, through the cannula body, and into the blood vessel in a first direction (e.g., towards the subject's heart). The proximal hub may include a second opening 140 extending at least partially from the distal end towards the proximal end. The second opening may be offset from the first opening. In some embodiments, a central axis 155 of the first opening is offset from a central axis 150 of the second opening by a predetermined distance 145. In some embodiments, the second opening extends all the way from the distal end to the proximal end. In some embodiments, the second opening may include, e.g., a membrane or hemostasis valve. In some embodiments, the first opening may have a larger diameter at the proximal end, such that the second opening connects to the first opening within the proximal hub at a point between the distal end and the proximal end.

In some embodiments, the second opening may form a hole, channel, or guide in the hub that points, along a path (such as a linear path extending along the central axis 150 of the second opening), towards the blood vessel so as to be targeting a second entry point 160 different from the entry point 112 of the distal cannula body. This second entry point can allow, e.g., a second cannula to be extended through the second opening, through the second entry point, and be directed into the blood vessel in a second direction different from the first direction (e.g., away from the subject's heart).

In some embodiments, the path will be substantially parallel to a proximal end 115 of the cannula body. In some embodiments, the path will be parallel to the central axis 155 of the first opening.

Alternatively, referring to FIG. 3, rather than having the guide or channel extend through the hub, in some embodiments, an accessory 200 (e.g., an add-on) may be provided for access to the second entry point. The accessory may be configured to attach to a proximal end 115 of the distal cannula body 110 or to a proximal hub 205 coupled to the cannula body. The accessory may include a separate cannula body 210 operably coupled to the distal cannula body 110 or proximal hub 205. For example, the add on may be clamped, snaped, glued, or otherwise coupled to the distal cannular body or proximal hub. The separate cannula body may be configured to point, along a path 220 (e.g., a linear path) towards the blood vessel so as to be targeting the second entry point 160. In still other embodiments, rather than a separate cannula body to provide such access to the second entry point, the distal cannula body may have a larger diameter at a proximal end than the distal end, the proximal end including a lumen that does not extend the entire length of the distal cannula body.

In some embodiments, the accessory may be removably coupled to the distal cannula body and/or proximal hub. In some embodiments, the accessory add-on may be permanently coupled to the distal cannula body and/or proximal hub.

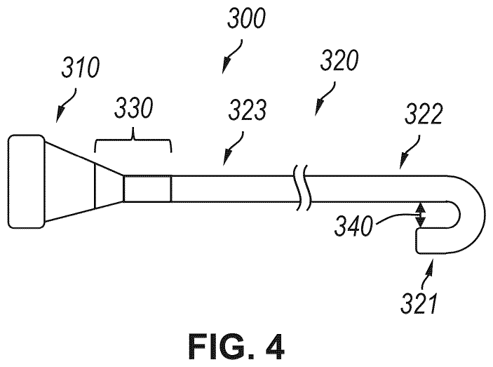

Referring to FIG. 4, in some embodiments, a device 300 may be provided having a proximal hub 310 coupled to a cannula body 320. In such embodiments, device may include an adjustable portion 330 that includes a distal end of the proximal hub and/or a proximal end 323 of the cannula body. In some embodiments, the adjustable portion is located within the distal end of the proximal hub. In some embodiments, the adjustable portion is coupled between the distal end of the proximal hub and the proximal end of the cannula body. In some embodiments, the adjustable portion may include an articulating joint, such as a ball joint, that is flexible and adjustable. The adjustable portion may allow for only 1-D rotation (e.g., rotation within a single plane) or 2-D rotation.

In some embodiments, the distal end 321 of the cannula body may be oriented to face a proximal direction. For example, the cannula body may be curved such that the distal end is parallel to a middle portion 322, but oriented in a 180 degree opposite direction. In some embodiments, an outer surface of the distal end 321 after a 180-degree bend is separated from an outer surface of the middle portion by a distance 340 of 0.25-1 mm. As will be appreciated, such a device may allow a physician flexibility in the direction in which access is achieved into the patient's vasculature.

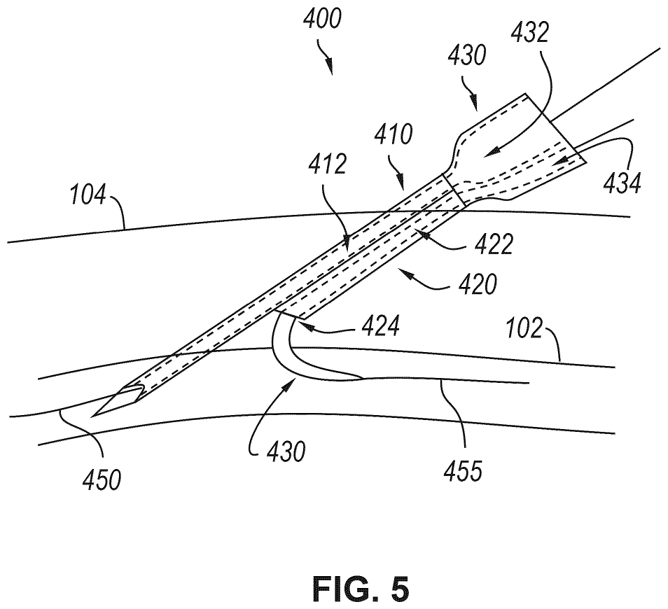

Referring to FIG. 5, in some embodiments, an introduction sheath 400 (sometimes referred to as an “introducer” or an “introducer sheath”) may be configured for multiple access into the vasculature. As shown in this view, the sheath 400 may include a tubular body 410. The tubular body 410 may include a first lumen 412 providing a first path extending from a proximal end of the tubular body to a distal end of the tubular body. The distal end of the tubular body may be configured to produce a micropuncture through the blood vessel. In some embodiments, the distal end of the tubular body is configured have an outer diameter of no more than 21 Fr.

The tubular body may include a proximal portion 420 defining a second lumen 422 providing a second path extending from the proximal end of the tubular body and extending distally. The second lumen may have an opening 424 at a distal end of the proximal portion that is configured to be positioned outside the blood vessel into which the first lumen is inserted. A curved needle or wire 455 may configured to be slidably received by the second lumen, such that the blood vessel may be entered at a different location from where the first lumen enters the blood vessel.

The introducer may include a hub 430 which may include one or more luer connections. In some embodiments, the hub may include two luer connections, one operably coupled to each lumen in the tubular body. The hub may include a first opening 432 configured to be coupled to a proximal end of the first lumen. The hub also may include a second opening 434 configured to be coupled to a proximal end of the second lumen. The first opening may be configured to slidably receive one or more medical devices configured to be inserted in a first direction (e.g., retrograde), and the second opening may be configured to slidably receive one or more medical devices configured to be inserted in a second direction different from the first direction (e.g., antegrade). In some embodiments, wires 450, 455 may be inserted retrograde and/or antegrade through an appropriate lumen.

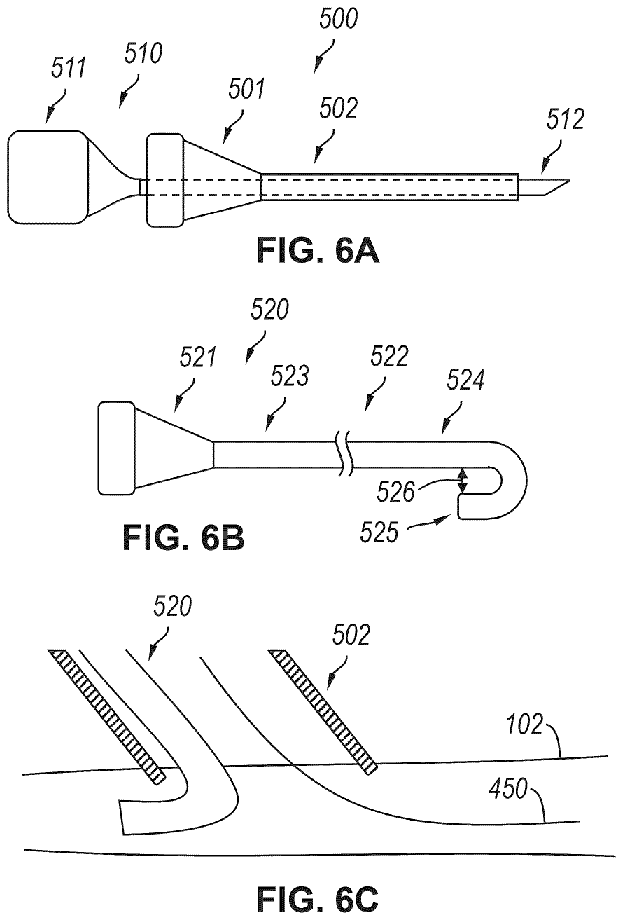

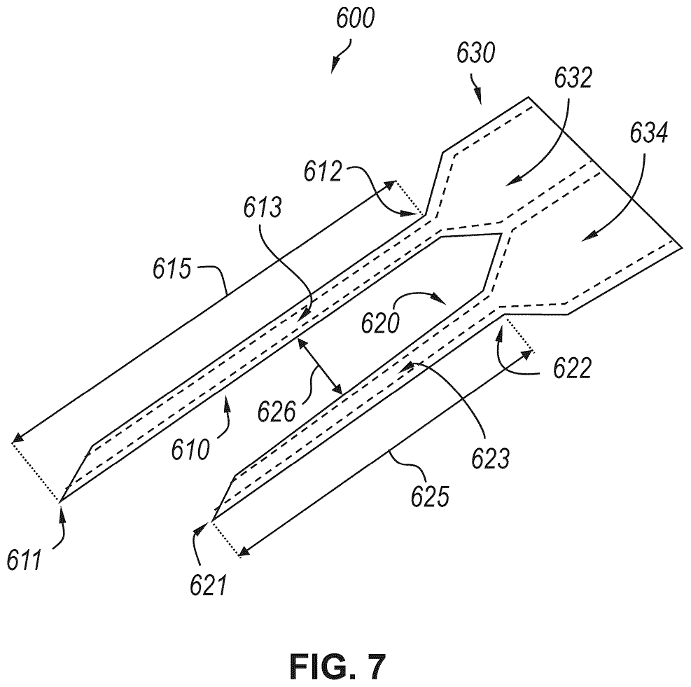

The needle tip is configured to puncture a blood vessel 102. In some embodiments, the second device 520 may be inserted into the catheter 510 after the needle has been removed. As shown in FIG. 6C, the distal end of the second device maybe configured to face a first direction (e.g., antegrade) while another device (e.g., a guide wire 450) is inserted in a second direction (e.g., retrograde).

In some embodiments, a large needle may be used in conjunction with one or more tubular members to provide access into the vasculature. Referring to FIGS. 6A-6C, a catheter 500 having a hub 501 coupled to a distal tubular member 502 may be configured to slidably receive a large needle 510. The large needle may include a hub 511 or handle portion, and a distal needle tip 512 configured to extend through the catheter. Separately, a second device 520 may be provided, the device including a proximal hub 521 coupled to a proximal end 523 of a distal tubular member 522, which may include a distal tip 525 that has curved to face a different direction from a middle portion 524 of the distal tubular member 522. The distal tip may have a fixed or variable separation from the middle portion.

It is recognized that, instead of using two devices, a single assembled device can be utilized, where the introducer tip can curve up to antegrade once a needle is removed. For example, instead of using a micro-puncture introducer (such as seen in FIG. 6A) with a second device (scc FIG. 6B) into the catheter, the single assembled device may have a catheter similar in shape to catheter 520 (in FIG. 6B). The needle may be pre-assembled into the catheter to straighten up the tip for users, so they can gain access using the assembly directly. Once they gain access, the needle (e.g., ref. 511/512) may be removed, and the catheter tip will curve to the antegrade direction. The user can then place the guidewire antegrade through the catheter.

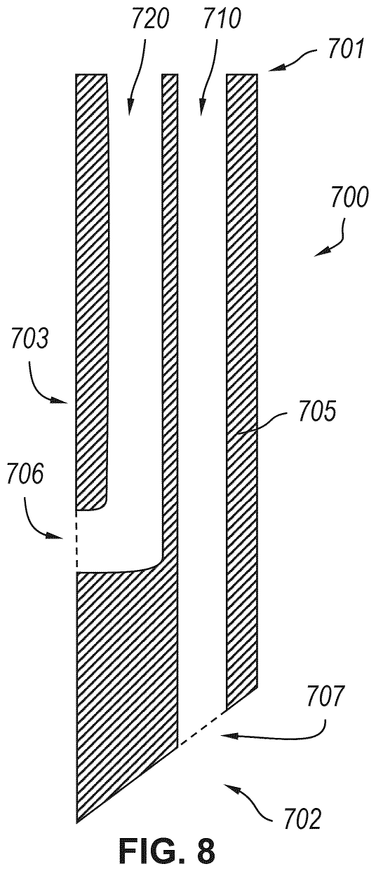

In some embodiments, a twin needle may be provided as a device for puncturing a blood vessel at multiple locations (see FIG. 7). One of the two needles may be useable for providing an access point for an antegrade sheath, and one of the two needles may be useable for providing an access point for a retrograde sheath. Referring to FIG. 7, a device 600 may be provided that includes a first needle 610. The first needle may define a lumen 613 extending from a distal end 611 to a proximal end 612. The first needle may have a first length 615, the length being from the distal end to the proximal end.

The device may include a second needle 620. The second needle may define a lumen 623 extending from a distal end 621 to a proximal end 622. The second needle may have a second length 625, the length being from the distal end to the proximal end. The second length may be shorter than the first length. As will be appreciated, the first and second needles may have any suitable length in other embodiments.

An outer surface of the first needle may be separated from an outer surface of the second needle by a fixed distance 626.

Each needle may be connected to a proximal hub 630 or handle. The proximal hub or handle may include a first opening 632 extending from a proximal end to the distal end of the hub, configured to be operably coupled to the lumen in the first needle. The proximal hub or handle may include a second opening 634 extending from a proximal end to the distal end of the hub, configured to be operably coupled to the lumen in the second needle. The openings and lumen may be configured such that one or more medical devices (e.g., guidewire, etc.) may be passed through the openings, through the lumen, and into the blood vessel.

In some embodiments, as an alternative or in combination with other devices disclosed herein, a needle with two lumens may be provided. Referring to FIG. 8, a cross-section of a needle can be seen. The needle 700 may include one or more sidewalls 705 that may define two lumen 710, 720, extending from a proximal end 701 towards a distal end 702 of the needle. A first lumen 710 may extend from the proximal end to the distal end, exiting at an opening 707 in the distal end of the needle. A second lumen 720 may extend from the proximal end but does not extend to the distal end. Rather, it passes through a sidewall in an intermediate portion 703 of the needle between the distal end and the proximal end, exiting through the sidewall at a second opening 706. The second opening 706 should be positioned sufficiently near the distal end such that, in use, the second opening is positioned within the blood vessel.

Using such a configuration, a single needle stick using a single access point (e.g., a same arteriotomy), can provide antegrade and retrograde access.

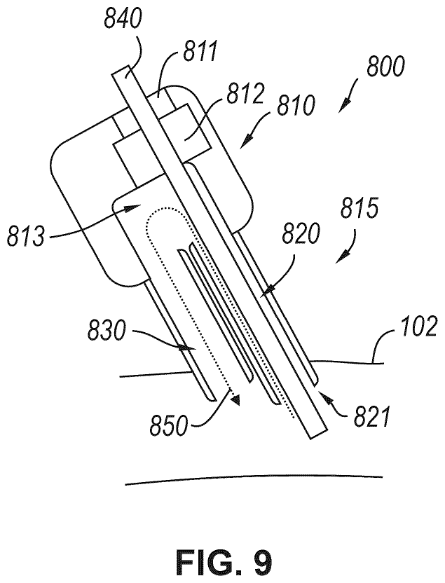

Referring to FIG. 9, in some embodiments, a device may operably couple two lumens together, e.g., for accessing a blood vessel without pressure. This may allow, e.g., blood to flow into a distal end of a first lumen and out through the distal end of the second lumen, into the blood vessel. Similarly, this may allow a wire to be directed in a secondary direction. As will be understood, this may be used with devices with two lumens, including, e.g., a dual lumen needle.

As seen in FIG. 9, the device 800 may include a hub 810 coupled to at least one tubular member 815 defining a first lumen 820 and a second lumen 830, the tubular member(s) extending distally away from the hub. The hub may include one or more valves 811, 812, and the first lumen 820 may be configured to slidably receive a medical device that passes through any valve(s). As shown, the hub may include a volume of space 813 coupled to both lumen, such that a path 850 from a distal opening for the first lumen to a distal opening for the second lumen can be formed by passing through the volume of space. Preferably, the first lumen is configured such that there is excess space within the first lumen after a medical device has been inserted, to allow, e.g., blood or a wire to pass through on the outside of the medical device.

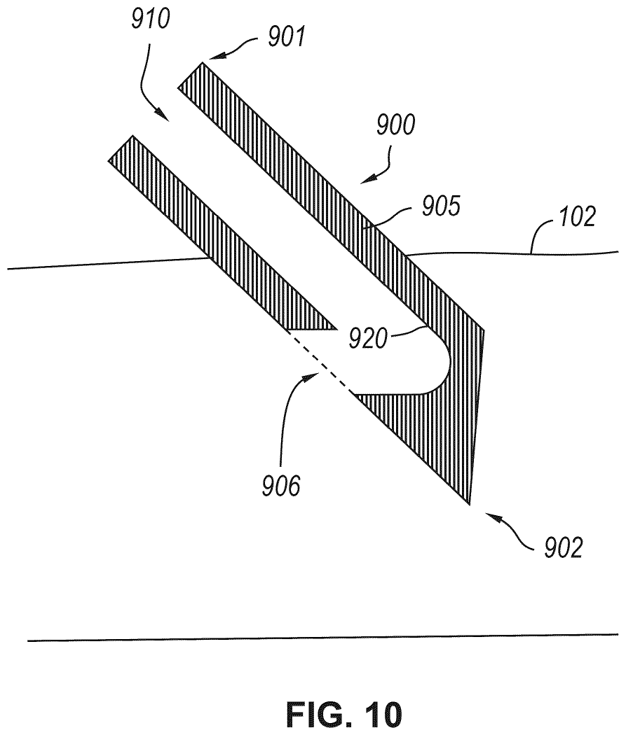

In some embodiments, a reverse needle may be provided. For purposes herein, a reverse needle refers to a needle configured to be inserted in a first direction (e.g., retrograde), but where a single outlet for the needle is configured to face a second direction (e.g., antegrade). Referring to FIG. 10, an embodiment of such a device may be seen. The reverse needle 900 may include one or more sidewalls 905 that define a lumen 910 extending from a proximal end 901 towards a distal end 902. The first lumen is shaped so as to exit via an opening 906 on a side of the needle (rather than an end), similar to the second lumen shown in FIG. 8. As will be appreciated, in such embodiments, the lumen may include a contact surface 920 which may direct the needle in the first direction (e.g., towards the distal end 902), and then cause it to move in a second direction (e.g., away from the distal end) and towards the sidewall opening 906).

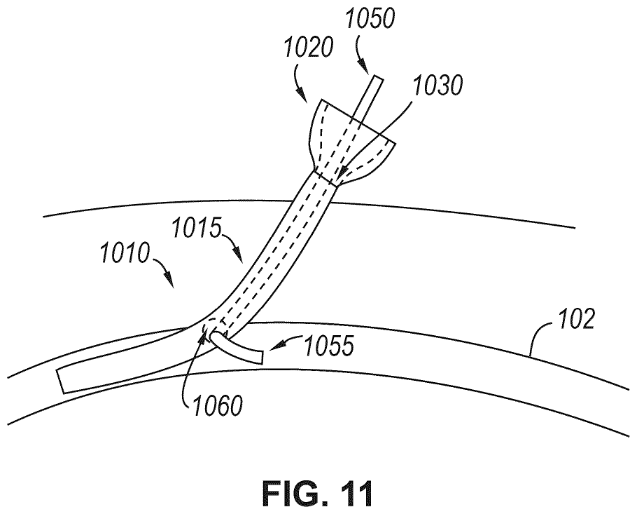

In some embodiments, a puncturable tubular member (such as a sheath) may be provided. For example, as seen in FIG. 11, a tubular member 1010 may have a proximal end coupled to distal end of a proximal hub 1020. The tubular member may have one or more arcas 1060 of the tubular member (but not the entire area) that may be puncturable (e.g., by a distal end of a medical device). In some embodiments the puncturable area may be free of a reinforcing coil. The puncturable arca also may have a sidewall thickness that is thinner than a sidewall thickness at a different location. The puncturable area may further be laser cut, and then may be filled with, e.g., a polymer. In some embodiments, a polymer forming the puncturable arca may be different from a polymer forming a sidewall in a different location. The puncturable area may have other suitable arrangements in other embodiments.

The puncturable area may be in a portion 1015 of the tubular member, such that in use, when the tubular member is positioned correctly, the puncturable area is within a blood vessel. In some embodiments, this portion 1015 may be located between a distal and proximal end of the tubular member.

The puncturable area may be configured such that an additional medical device 1050 (e.g., a cannula, a wire, etc.) may be slidably received by the hub, passing through an opening 1030 in the hub and into the tubular member, and a distal end 1055 of an additional medical device may be configured to pass through the puncturable area and extend in a opposite direction in the blood vessel compared to the direction the tubular member extends (e.g., extending antegrade when the tubular member extends antegrade).

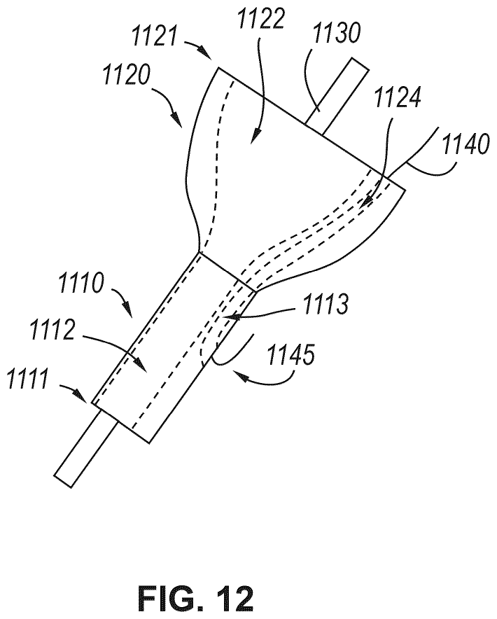

Referring to FIG. 12, in some embodiments, a device may be provided where a tubular member 1110 may be coupled to a proximal hub 1120. The tubular member may include two lumens. A first lumen 1112 may extend from the hub to a distal end 1111. A second lumen 1113 may extend from the hub and exit a side opening. As shown, a medical device may be slidably received through a first opening 1122 in the hub operably connected to the first lumen. A second medical device (e.g., a guide wire, a needle, etc.) may be slidably received through a second opening 1124 in the hub operably connected to the second lumen. At least a portion 1145 of the second medical device may have a memory shape, such that when it exits out an opening through a sidewall of the tubular member, it returns to the shape, e.g., a J-shape.

In some embodiments, the device may be configured such that, in use, when the tubular member is correctly placed, the opening through the sidewall is positioned within a blood vessel.

In some embodiments, the tubular member may include an expandable sleeve, e.g., a polymeric sleeve, through which the second medical device can be inserted.

In various embodiments, the tubular member may be a catheter, cannula, or needle.

In some embodiments, a sheath and/or sleeve may be provided over or around any needle.

During insertion using a needle and catheter, when the patient experiences bleedback, the physician may advance the catheter. The physician may then put in a guidewire, which can be used with or for, e.g., a kink-resistant sheath.

The embodiments with dual lumen allow for one lumen to be used to provide retrograde access, and one lumen to provide antegrade access. When providing antegrade access, in some embodiments, the lumen may be shaped or configured to reduce the resistance to the change of direction required for such insertions. For example, sharp angle bends are typically disfavored over gentler curves or bends.

In some embodiments, the various embodiments may include one or more attachments for needles. For example, in some embodiments, a clutch may be provided that applies spring force/tension in a forward direction so that as one enters a blood vessel with, e.g., a guidewire that includes the needle, if there is vacant space, the guidewire will drop forward slightly. This may be used in a deflated vessel (e.g., a limb that is already ischemic). Such attachments may provide casier access and feedback for proper placement without using ultrasound. In some embodiments, such attachments could be used with a probe, rather than a wire.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

1. A retrograde sheath, comprising:
a hub comprising a first opening extending from a proximal end to a distal end of the hub, and a second opening extending from the proximal end to the distal end of the hub; and
a first cannula coupled to a distal end of a hub, the first opening being operably coupled to the first cannula.

2. The retrograde sheath of claim 1, wherein the second opening includes a membrane or hemostasis valve.

3. The retrograde sheath of claim 1, wherein the first opening has a larger diameter at the proximal end, such that the second opening connects to the first opening within the hub at a point between the distal end and the proximal end.

4. The retrograde sheath of claim 1, wherein the first opening is configured to be directed towards a blood vessel, and the second opening is configured to be directed towards the blood vessel.

5. The retrograde sheath of claim 4, wherein the first cannula is configured to be directed into the blood vessel in a first direction, and a second cannula is configured to be directed into the blood vessel in a second direction different from the first direction.

6. The retrograde sheath of claim 1, wherein the first cannula has a larger diameter at a proximal end than a distal end, the proximal end including a first lumen that extends towards the distal end that extends a first length, and a second lumen parallel to the first lumen that extends towards the distal end a distance less than the first length.

7. A retrograde sheath, comprising:
a hub comprising a first opening extending from a proximal end to a distal end of the hub;
a distal cannula having a distal cannula body coupled to a distal end of a hub, the first opening being operably coupled to the distal cannula; and
an accessory coupled to a proximal end of the distal cannula body or to the hub, the accessory including a separate cannula body.

8. The retrograde sheath of claim 7, wherein the distal cannula is configured to be directed towards a blood vessel, and the separate cannula body is configured to point towards the blood vessel.

9. The retrograde sheath of claim 8, wherein the accessory is removably coupled to the distal cannula body or the hub.

10. The retrograde sheath of claim 8, wherein the accessory is permanently coupled to the distal cannula body or the hub.

11. A retrograde sheath, comprising:
a proximal hub coupled to a cannula body,
wherein the retrograde sheath includes an adjustable portion, the adjustable portion including a distal end of the proximal hub and/or a proximal end of the cannula body.

12. The retrograde sheath of claim 11, wherein the adjustable portion is located within the distal end of the proximal hub.

13. The retrograde sheath of claim 11, wherein the adjustable portion is coupled between the distal end of the proximal hub and the proximal end of the cannula body.

14. The retrograde sheath of claim 11, wherein the adjustable portion includes an articulating joint.

15. The retrograde sheath of claim 11, wherein the adjustable portion is configured to allow only 1-D rotation.

16. The retrograde sheath of claim 11, wherein the adjustable portion is configured to allow only 1-D or 2-D rotation.

17. The retrograde sheath of claim 11, wherein a distal end of the cannula body is oriented to face a proximal direction.

18. The retrograde sheath of claim 11, wherein an outer surface of a distal end of the cannula body, after a 180-degree bend, is separated from an outer surface of a middle portion of the cannula body by a distance of 0.25-1 mm.

19-41. (canceled)

42. A device, comprising:
a hub coupled to at least one tubular member defining a first lumen and a second lumen, where the at least one tubular member extends distally away from the hub, the first lumen configured to slidably receive a medical device; and
one or more valves disposed within the hub;
wherein the hub defines a volume of space coupled to the first lumen and the second lumen, such that a path from a distal opening for the first lumen to a distal opening for the second lumen can be formed by passing through the volume of space.

43-61. (canceled)
